(19) United States
(12) Patent Application Publication
(10) Pub. No.: US 2016/0310278 A1
(43) Pub. Date: Oct. 27, 2016

(54) JAWBONE TRANSPLANT ARRANGEMENT

(71) Applicant: Peter KESSLER, Nidderau (DE)

(72) Inventor: Peter KESSLER, Nidderau (DE)

(21) Appl. No.: 15/138,613

(22) Filed: Apr. 26, 2016

(30) Foreign Application Priority Data
Apr. 27, 2015 (EP) 15165160.1

(51) Int. Cl. A61F 2/28

(52) U.S. Cl. CPC A61F 2/2803; A61F 2310/00359

(57) ABSTRACT

Jawbone transplant arrangement and method of forming jawbone transplant arrangement. The jawbone transplant arrangement includes a bone transplant that has at least one first freeform surface and at least one implant opening.

### CROSS-REFERENCE TO RELATED APPLICATION

The present application claims priority under 35 U.S.C. §119 of European Patent Application No. 15 165 160.1, filed Apr. 27, 2015, the disclosure of which is expressly incorporated by reference herein in its entirety.

### BACKGROUND

1. Field of the Invention

Embodiments of the invention relate to a jawbone transplant arrangement with a jawbone transplant.

2. Discussion of Background Information

One consequence of diseases or treatments of a jawbone can be the loss of jawbone matrix. This loss can occur, e.g., after a tooth extraction or cystectomy. It can also appear in the course of bone atrophy as a consequence of tooth loss or inflammatory processes. Various options are then available for replacing and for regenerating the lost jawbone matrix.

One option is jawbone augmentation with an autologous (a patient's own) bone transplant. The use of an autologous bone transplant is advantageous because of the biological activity thereof, which is determined by vital cells and growth factors. The autologous bone transplant can be obtained in intraoral donor regions of the patient. However, in this case the availability of sufficient quantities is limited. The removal of other autologous bone, for example from the iliac crest of the patient, requires an additional operative site. An additional bone defect is produced with potential morbidity at the removal site.

An alternative to using an autologous bone transplant is the application of processed allogeneic (bone from human donors) bone transplants. Allogeneic bone transplants of this type can be individually adapted to the jawbone defect of the patient. For this purpose, a corresponding bone transplant is virtually designed with the aid of planning software, and the jawbone defect in question reconstructed. As a result of the data record created in this manner, an individual bone transplant can be milled.

The bone transplant produced can then be fitted into the jawbone defect by the treating physician. It is fixed to the jawbone, for example using osteosynthesis screws. Finally, the bone transplant is covered with bone replacement material and a collagen membrane. The collagen membrane thereby serves as a resorption barrier.

The trabecular structure of the bone transplant enables a rapid uptake of blood and nutrients. The process of incorporating the bone transplant exhibits a high reliability and predictability.

After completion of the incorporation of the bone transplant, a further operative step can take place. In this step, the soft tissue surrounding the bone transplant can be prepared such that the treating physician has access to the bone transplant. Implant openings can then be introduced in the bone transplant, for example by drilling. Implants can be inserted into the implant openings. The implants can be used to fix and position, e.g., a dental prosthesis.

The introduction of implant openings in the bone transplant after an already completed incorporation process is, however, disadvantageous insofar as it can result in a weakening of the primary stability of the bone transplant and implant. Furthermore, the patient is also stressed by an additional extensive surgical intervention. The healing time of the bone transplant is thus significantly extended.

### SUMMARY

Embodiments of the invention provide a jawbone transplant arrangement that has a short healing time.

In embodiments, a jawbone transplant arrangement of the type named at the outset includes a bone transplant having at least one first freeform surface and at least one implant opening.

The first freeform surface is matched to a jawbone defect of a patient. Within the context of this disclosure a freeform surface can be understood as a surface that as a whole cannot be represented by known geometric structures. Known geometric structures of this type are, e.g., circles, ellipses, triangles, rectangles, etc. The freeform surface can be, e.g., modeled by the use of appropriate planning software and the three-dimensional visualization of the jawbone defect. The bone transplant can be individually milled with the aid of the data obtained in this manner. The implant opening can be used for inserting implants. A position of the implant opening in the bone transplant can also be determined and defined with the planning software. The implant openings can be introduced in the bone transplant in the milling process. Thus, it is possible to perform an augmentation of the jawbone by the bone transplant with a simultaneous implantation. In contrast to the two-phase augmentation of the jawbone described above, the entire treatment time can now be significantly reduced. A second extensive surgical intervention can be avoided. The healing time of the bone transplant can be reduced overall. The surgical treatment of three-dimensional jawbone defects is simplified.

It is thereby preferred that the bone transplant includes a second freeform surface, or a plane, that encloses a freeform volume with the first freeform surface. The freeform volume determines the extent of the augmentation of the jawbone. The freeform in volume can thereby be matched to the conditions of the surrounding jawbone by an adaptation of the second freeform surface or the plane. With regard to the jawbone, both a horizontal and also a vertical augmentation of the jawbone are possible.

It is preferred that the implant opening is embodied or formed on a side of the bone transplant opposite the first freeform surface. The first freeform surface is, in terms of its shape, adapted to the shape of the jawbone defect. The side of the bone transplant opposite the first freeform surface is framed by the second freeform surface or the plane. On this side of the bone transplant, the implants to which a dental prosthesis can be attached are to be inserted. The conditions that the treating physician encounters on the jawbone of the patient determine whether this side of the bone transplant is embodied or formed as a second freeform surface or as a plane. The side of the bone transplant in question is thereby embodied or formed as a plane if, for example, the position of the provided dental prosthesis permits it. The embodiment of a plane on the bone transplant is easier to produce than the second freeform surface.

Preferably, the implant opening is arranged in the freeform volume and embodied or formed as a through-hole or blind hole. The embodiment of the implant opening as a through-hole allows the attachment of the bone transplant to the jawbone. For this purpose, osteosynthesis screws for example can be accommodated in the implant opening and screwed to the jawbone. The osteosynthesis screws are then accommodated in the freeform volume. However, it is equally possible to embody or form the implant opening as a blind hole. This is for example conceivable if a less invasive type of attachment of the bone transplant to the jawbone is possible. In the event that the implant opening is embodied or formed as a through-hole, the implant opening connects the first freeform in surface to the second freeform surface or the plane. It is possible to create the through-hole or the blind hole, e.g., via a drilled hole in the prefabricated bone transplant.

In all of this, it is preferred that the bone transplant includes at least two implant openings. In each of the at least two implant openings, one implant can be inserted. In this way, it is possible to use the jawbone transplant arrangement beyond the use for a single-tooth gap. A corresponding dental prosthesis can be attached to each of the implants that can be accommodated in the implant openings.

Preferably, the implant opening includes an attachment element that is engaged with the bone transplant, and to which an implant can be attached. The attachment element is fitted into the jawbone defect simultaneously with the bone transplant. It is achieved that, during the process of incorporating the bone transplant, no bone tissue forms in the implant opening. The implant opening thus also remains intact during the incorporation process and retains its provided nominal diameter. The attachment element does not have to be removed again once incorporation of the bone transplant is complete. It is used to attach the implant. Through the simultaneous introduction of the bone transplant together with the attachment element, a greater primary stability of the bone transplant and of the implant is achieved.

It is thereby preferred that the attachment element is formed from a biologically inactive material, wherein an axial extension of the attachment element is matched to an axial extension of the implant opening. The biologically inactive material of the attachment element does not take part in the process of incorporating the bone transplant. Furthermore, the biologically inactive material can be sterilized. Prior to a transplantation of the bone transplant with the attachment element, the attachment element is sterilized. In this manner, it is ensured that the attachment element is to a large extent pathogen-free so that an infection can be avoided. By matching the axial extension of the attachment element to the axial extension of the implant opening, it can be achieved that the attachment element is completely, or at least in large part, accommodated in the implant opening. If the attachment element is present, it is thus also possible to cover the bone transplant with a collagen membrane or surrounding soft tissue without problems.

It is also preferred that the attachment element is embodied or formed as a sleeve made from a metal alloy or a metal, in particular titanium. An implant can be easily inserted into a sleeve of this type. It is possible to match the sleeves used to the implants required. For example, standardized drill sleeves can be used. Drill sleeves of this type are preferably made of titanium. A metal or a metal alloy is thereby biologically inactive and easy to sterilize.

It is also preferred that a position of the implant opening in the bone transplant is matched to a position of a dental prosthesis relative to the bone transplant. The position of the implant opening in the bone transplant is thereby chosen such that the dental prosthesis can be easily integrated into the surrounding environment of the bone transplant. The planning of the position of the implant opening in the bone transplant can thereby already occur during the planning of the bone transplant and the intended augmentation.

Finally, it is preferred that the dental prosthesis can be attached to the implant. It is thus achieved that, after the completed process of incorporating the bone transplant and a further removal of the surrounding soft tissue in a second operation step, the provided dental prosthesis can be easily attached to the implant already located in the bone transplant. A larger invasive intervention can be avoided. In this manner, the healing time of the jawbone transplant arrangement can be reduced.

Embodiments of the invention are directed to a jawbone transplant arrangement that includes a bone transplant that has at least one first freeform surface and at least one implant opening.

According to embodiments, the bone transplant may further have one of at least one second freeform surface or at least one planar surface that with the first freeform surface encloses a freeform volume. The at least one second freeform surface can include a planar portion.

In accordance with other embodiments, the implant opening can be formed on a surface of the bone transplant opposite the first freeform surface.

According to other embodiments, the implant opening may be arranged in the freeform volume and can include one of a through-hole or blind hole.

In other embodiments of the invention, the at least one implant opening may include at least two implant openings.

According to still other embodiments, the implant opening can include an attachment element that is engaged with the bone transplant and to which an implant can be attached. The attachment element can be formed from a biologically inactive material. Further, the attachment element may be embodied as a sleeve made from a metal alloy or a metal, in particular titanium. Moreover, an axial extension of the attachment element can be matched to an axial extension of the implant opening.

In accordance with other embodiments, a position of the implant opening can be matched to a position of a dental prosthesis relative to the bone transplant. The dental prosthesis can be attached to the implant.

Embodiments of the instant invention are directed to a method of forming the above-described jawbone transplant arrangement. The method includes creating a virtual three-dimensional planning model of a jawbone defect; determining a structure of the bone transplant based upon the virtual three-dimensional planning model; and producing the bone transplant from a block of bone based on the determined structure.

According to embodiments, the bone transplant can be produced by milling the block of bone.

In accordance with still other embodiments of the present invention, the produced bone transplant can include a freeform volume that is delimited by the first freeform surface and one of at least one second freeform surface or at least one planar surface, and the implant opening. The second freeform surface can include a planar portion. The method can also include forming the implant opening in the one of at least one second freeform surface or at least one planar surface. The method can also include forming the implant opening as one of a through hole and a blind hole. In embodiments, the method can also include arranging an attachment element in the implant opening. The attachment element can be formed from a material comprising metal or metal alloy.

Other exemplary embodiments and advantages of the present invention may be ascertained by reviewing the present disclosure and the accompanying drawings.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a jawbone transplant arrangement with a bone transplant which comprises an implant opening;

FIG. 2 shows a further illustration of the jawbone transplant arrangement from FIG. 1; and

FIG. 3 shows a second jawbone transplant arrangement with three implant openings.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

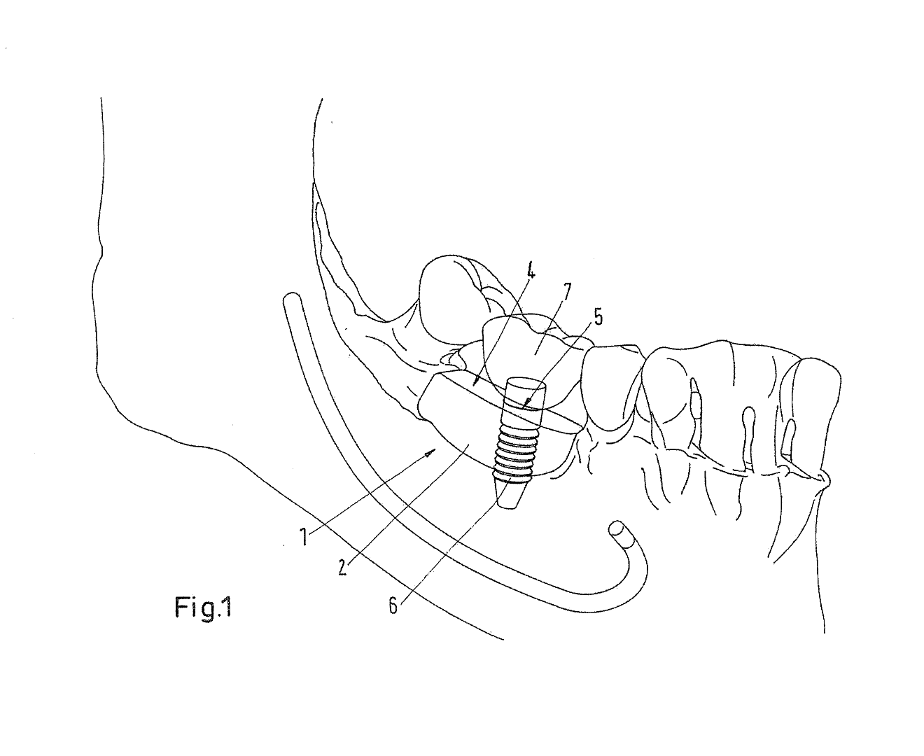

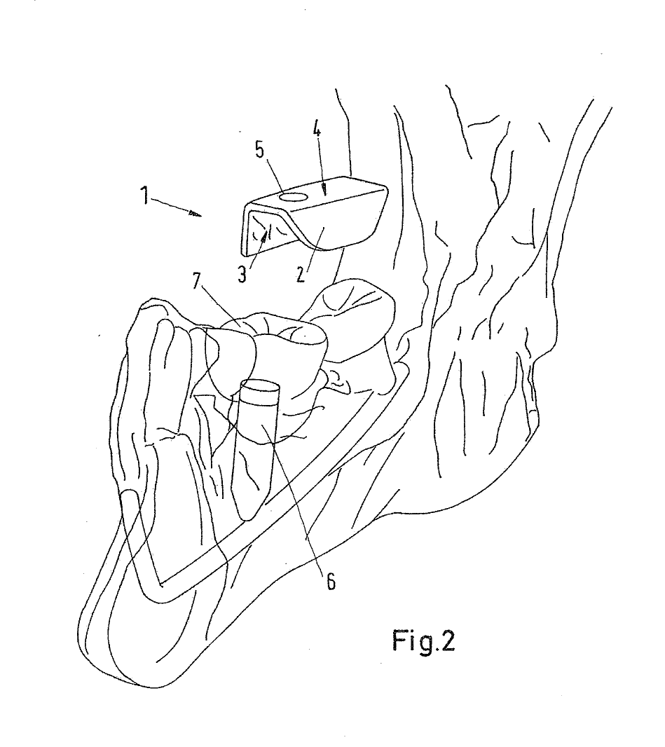

FIG. 1 shows a jawbone transplant arrangement 1 with a bone transplant 2. The jawbone transplant arrangement 1 comprises a freeform volume that is enclosed by a first freeform surface 3 and a second freeform surface, such as a plane 4. On the plane 4, a first implant opening 5 is arranged. In the first implant opening 5, an implant 6 is accommodated. For this purpose, the implant opening 5 is embodied or formed as a through-hole. In the exemplary embodiment in FIGS. 1 and 2, the implant 6 is an osteosynthesis screw. A dental prosthesis 7 is attached to the implant 6.

Particularly in FIG. 2, it can be seen that the first freeform surface 3 is matched to a jawbone defect. In this way, a more precise fit of the bone transplant 2 to the jawbone is thus rendered possible. The side of the bone transplant 2 opposite the first freeform surface 3 is embodied or formed by a second freeform surface, which can include a planar surface 4, which is possible and is preferred due to the structure of the jawbone defect. The first freeform surface 3 and the plane 4 enclose the freeform volume. This freeform volume defines the augmentation that is provided by the bone transplant 2.

The production of the bone transplant 2 can occur in the following steps. In a first step, a virtual three-dimensional planning model of the jawbone defect is created. With planning software, the structure of the bone transplant 2 is then determined. The bone transplant 2 can subsequently be produced in a milling process from a block of bone. The structure of the bone transplant 2 is thereby determined by the first freeform surface 3, the second freeform surface 4, which can include a planar surface, and the implant opening 5.

Before the bone transplant 2 is transplanted into the jawbone of the patient, an attachment element (not illustrated) can be arranged and accommodated in the implant opening 5. For this purpose, the implant opening 5 is embodied or formed as a through-hole or blind hole.

The attachment element is engaged with the bone transplant 2. The implant 6 can be attached thereto. The attachment element is thereby preferably formed from a biologically inactive material. In this manner, on the one hand, the occurrence of a closure of the implant opening by bone tissue during the process of incorporating the bone transplant 2 can be avoided so that the implant opening 5 can be kept at its provided diameter. On the other hand, the attachment element will be formed from a sterile or sterilizable material so that infections of the bone transplant 2 can thus be avoided.

In order to enable a covering of the bone transplant 2 with a collagen membrane or surrounding soft tissue, an axial extension of the attachment element is matched to an axial extension of the implant opening 5. The attachment element can thus for example be embodied or formed such that it can be completely, or at least in large part, accommodated in the implant opening 5.

It is thereby particularly preferred that the attachment element is embodied or formed as a sleeve. This sleeve can have standardized dimensions. It can for example be embodied as a drill sleeve. The drill sleeve can thereby be formed from a metal or a metal alloy. For example, titanium can be used as the metal. A sleeve made of a metal or a metal alloy is thereby easily sterilized.

Furthermore, a position of the implant opening 5 in the bone transplant 2 is matched to a position of the dental prosthesis 7 relative to the bone transplant 2. The dental prosthesis 7 can thus be integrated into the surrounding teeth and the surrounding jawbone structure in a precisely fitting manner. The dental prosthesis 7 can be attached to the implant 6.

The augmentation of the jawbone can then be performed simultaneously with the implantation. As a result, the patient's treatment time can be significantly reduced. A further intervention to create the implant opening 5 and to introduce the implant 6 after the completed process of incorporating the bone transplant 2 can be omitted. Because of the simultaneous introduction of the bone transplant 2 and the implant 6, a greater primary stability of the bone transplant 2 and implant 6 is achieved. A simplified surgical treatment of three-dimensional jawbone defects can be achieved. The reliability in everyday clinical use of the jawbone transplant arrangement 1 according to the invention is increased and the healing times for the jawbone transplant arrangement 1 can be reduced.

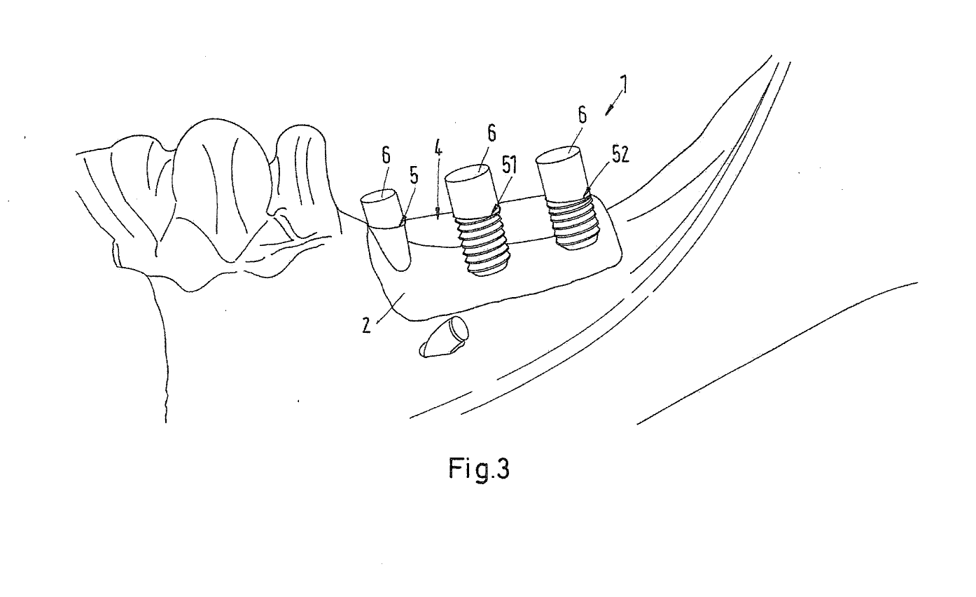

FIG. 3 shows a jawbone transplant arrangement 1 with a first implant opening 5, a second implant opening 51 and a third implant opening 52. Otherwise, identical technical features are provided with identical reference numerals.

In addition to an individual tooth gap, the jawbone transplant arrangement 1 can also be used for the horizontal and vertical augmentation of the jawbone that is to encompass more than one dental prosthesis 7.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A jawbone transplant arrangement comprising:
a bone transplant that includes at least one first freeform surface and at least one implant opening.

2. The jawbone transplant arrangement according to claim 1, wherein the bone transplant further includes one of at least one second freeform surface or at least one planar surface that with the first freeform surface encloses a freeform volume.

3. The jawbone transplant arrangement according to claim 2, wherein the at least one second freeform surface comprises a planar portion.

4. The jawbone transplant arrangement according to claim 1, wherein the implant opening is formed on a surface of the bone transplant opposite the first freeform surface.

5. The jawbone transplant arrangement according to claim 2, wherein the implant opening is arranged in the freeform volume and comprises one of a through-hole or blind hole.

6. The jawbone transplant arrangement according to claim 1, wherein the at least one implant opening comprises at least two implant openings.

7. The jawbone transplant arrangement according to claim 1, wherein the implant opening comprises an attachment element that is engaged with the bone transplant and to which an implant is attachable.

8. The jawbone transplant arrangement according to claim 7, wherein the attachment element is formed from a biologically inactive material.

9. The jawbone transplant arrangement according to claim 8, wherein the attachment element is embodied as a sleeve made from a metal alloy or a metal.

10. The jawbone transplant arrangement according to claim 9, wherein the metal alloy or metal comprises titanium.

11. The jawbone transplant arrangement according to claim 7, wherein an axial extension of the attachment element is matched to an axial extension of the implant opening.

12. The jawbone transplant arrangement according to claim 1, wherein a position of the implant opening is matched to a position of a dental prosthesis relative to the bone transplant.

13. The jawbone transplant arrangement according to claim 12, wherein the dental prosthesis is attachable to the implant.

14. A method of forming the jawbone transplant arrangement according to claim 1, the method comprising:
creating a virtual three-dimensional planning model of a jawbone defect;
determining a structure of the bone transplant based upon the virtual three-dimensional planning model; and
producing the bone transplant from a block of bone based on the determined structure.

15. The method according to claim 14, wherein the bone transplant is produced by milling the block of bone.

16. The method according to claim 14, wherein the produced bone transplant includes a freeform volume that is delimited by the first freeform surface and one of at least one second freeform surface or at least one planar surface, and the implant opening.

17. The method according to claim 16, wherein the second freeform surface comprises a planar portion.

18. The method according to claim 16, further comprising forming the implant opening in the one of at least one second freeform surface or at least one planar surface.

19. The method according to claim 18, further comprising forming the implant opening as one of a through hole and a blind hole.

20. The method according to claim 16, further comprising arranging an attachment element in the implant opening.
